(19) United States
(12) Patent Application Publication
(10) Pub. No.: US 2017/0143529 A1
(43) Pub. Date: May 25, 2017

(54) SPINE IMMOBILIZATION DEVICE

(71) Applicant: MUYU CO., LTD., Tokyo (JP)

(72) Inventor: Hiroshi ITO, Tokyo (JP)

(73) Assignee: MUYU CO., LTD., Tokyo (JP)

(21) Appl. No.: 15/402,841

(22) Filed: Jan. 10, 2017

Related U.S. Application Data
(63) Continuation of application No. PCT/JP2014/073158, filed on Sep. 3, 2014.

(51) Int. Cl. A61F 5/02

(52) U.S. Cl. CPC A61F 5/02; A61F 5/028

(57) ABSTRACT

Provided is a spine immobilization device that is able to suppress the sense of discomfort felt when worn to a low level. A spine immobilization device is an immobilization device that immobilizes a person's spine and is provided with a sheet part and a core part. The sheet part has a sheet-like shape and is configured to be stuck to the body surface. The core part is fastened to the back surface of the sheet part. The core part is configured to extend along a spine. In this state, the core part laterally supports the spine.

### CROSS REFERENCE TO RELATED APPLICATION

This is a Continuation of International Application No. PCT/JP2014/073158 filed Sep. 3, 2014. The contents of this application are hereby incorporated, by reference in their entirety.

### TECHNICAL FIELD

The present invention relates to a spine immobilization device that immobilizes a person's spine.

### BACKGROUND ART

Patent Literature 1 describes a corset that is worn around the waist. The corset described in this literature has a waist belt that is wrapped around the waist, a rear substrate that is fastened to a position of the waist belt that opposes the spine, and a reinforcing plate that is fastened to the rear substrate. When this corset is worn, the reinforcing plate is pressed against the spine by the clamping force of the waist belt.

### CITATION LIST

### Patent Literature

[PLT 1] JP 2009-34453A

### SUMMARY OF INVENTION

### Technical Problem

The corset described in Patent Literature 1 is able to immobilize the spine to a certain extent, by the reinforcing plate pressed against the spine. Therefore, this corset can also be used as a spine immobilization device for immobilizing the spine.

However, despite the spine being the region that is to be immobilized in the case of using this corset as a spine immobilization device, the whole circumference of the waist is covered by the corset. A large area of the body thus being covered by the spine immobilization device is a factor that intensifies the sense of discomfort caused by wearing the spine immobilization device.

### Solution to Problem

The present invention was made in view of the above problem, and has an object to provide a spine immobilization device that is able to suppress the sense of discomfort felt when worn to a low level.

A spine immobilization device according to the present invention is a spine immobilization device for immobilizing a spine of a person and includes a sheet part configured to be stuck to a body surface, and a core part configured to be fastened to the sheet part and to extend along the spine so as to laterally support the spine.

With this spine immobilization device, the spine can be immobilized by the core part that laterally supports the spine. Moreover, this spine immobilization device can be worn on the body by sticking the sheet part to the body surface. Therefore, the spine immobilization device can be worn, without being wrapped around the body. The area of the body that is covered by the spine immobilization device is thereby narrowly limited, enabling the sense of discomfort felt when the spine immobilization device is worn to be suppressed to a low level.

### Advantageous Effects of Invention

According to the present invention, a spine immobilization device that is able to suppress the sense of discomfort felt when worn to a low level is realized.

### BRIEF DESCRIPTION OF DRAWINGS

FIG. 1 is a plan view showing a first embodiment of a spine immobilization device according to the present invention.

FIG. 2 is a cross-sectional view along a line II-II of the spine immobilization device of FIG. 1.

FIG. 3 is a diagram for illustrating a state in which the spine immobilization device of FIG. 1 is worn on the body.

FIG. 4 is a diagram for illustrating the positional relationship between a core part in the spine immobilization device of FIG. 1 and the spine.

FIG. 5 is a diagram for illustrating that the core part in the spine immobilization device of FIG. 1 is deformable in the front-back direction of the body.

FIG. 6 is a diagram for illustrating that the core part in the spine immobilization device of FIG. 1 is not deformable in the right-left direction of the body.

FIG. 7 is a plan view showing a second embodiment of the spine immobilization device according to the present invention.

FIG. 8 is a diagram for illustrating a state in which the spine immobilization device of FIG. 7 is worn on the body.

FIG. 9 is a plan view showing a third embodiment of the spine immobilization device according to the present invention.

FIG. 10 is a plan view showing a fourth embodiment of the spine immobilization device according to the present invention.

FIG. 11 is a plan view showing a spine immobilization device according to a variation.

FIG. 12 is a cross-sectional view showing the core part according to a variation.

FIG. 13 is a plan view showing the core part according to another variation.

FIG. 14 is a diagram for illustrating movement of the core part in FIG. 13.

### DESCRIPTION OF EMBODIMENTS

Hereinafter, embodiments of the present invention will be described in detail with reference to the drawings. Note that, in the description of the drawings, like elements are denoted by like reference numerals, and redundant description is omitted.

### First Embodiment

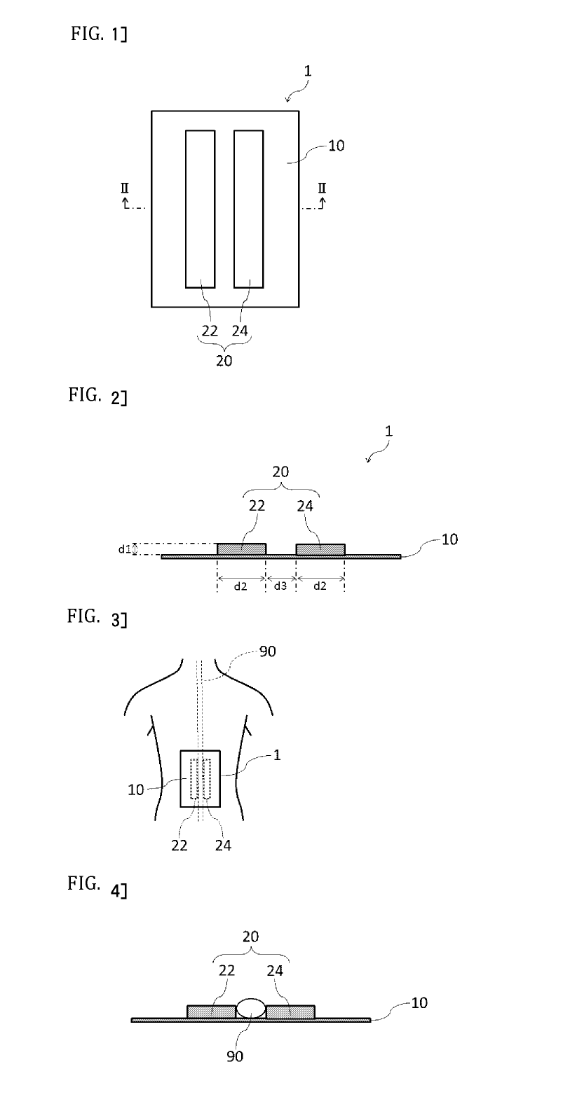

FIG. 1 is a plan view showing a first embodiment of a spine immobilization device according to the present invention. Also, FIG. 2 is a cross-sectional view along a line II-II of FIG. 1. A spine immobilization device 1 is an immobilization device that immobilizes a person's spine, and is provided with a sheet part 10 and a core part 20. In the present embodiment, the spine immobilization device 1 is for immobilizing the lumbar spine.

The sheet part 10 has a sheet-like shape and is configured to be stuck to the body surface. Specifically, the sheet part 10 is configured to be stuck to a region of the body surface near the spine (lumbar spine). Therefore, an adhesive that has adhesive power on the body surface is applied to the back surface (surface on the side facing the body surface) of the sheet part 10. Note that FIG. 1 shows the back surface side of the sheet part 10. The spine immobilization device 1 is worn on the body as a result of the sheet part 10 being stuck to the body surface. The sheet part 10 preferably can be repeatedly stuck to the body surface. Paper, woven fabric or nonwoven fabric, for example, can be used as the material of the sheet part 10. The sheet part 10 preferably has elasticity. Also, the sheet part 10 preferably has breathability. The sheet part 10 has, for example, a width (length in the right-left direction in FIG. 1) of about 15 cm and a length (length in the up-down direction in FIG. 1) of about 20 cm.

The core part 20 is fastened to the back surface of the sheet part 10. In the present embodiment, the core part 20 consists of a core part 22 (first core part) and a core part 24 (second core part). These core parts 22 and 24 are fastened to a single one of the sheet part 10. This fastening can, for example, be carried out by the adhesive power of the adhesive applied to the sheet part 10. Alternatively, the core parts 22 and 24 and the sheet part 10 may be fastened using a surface fastener. The core parts 22 and 24 are preferably attachable and detachable with respect to the sheet part 10. Here, being attachable and detachable means that the core parts 22 and 24 can be attached to or removed from the sheet part 10, without damaging the spine immobilization device 1.

The core parts 22 and 24 each have a rectangular columnar shape. The core parts 22 and 24 each preferably have a thickness d1 (refer to FIG. 2) from 2 mm to 5 mm inclusive. The core parts 22 and 24 each have a width d2 from 2 cm to 5 cm inclusive, for example. Also, the core parts 22 and 24 each preferably have a length of 15 cm or more. These core parts 22 and 24 extend parallel at a fixed interval d3 (refer to FIG. 2). The interval d3 is, for example, from 1 cm to 3 cm inclusive. This interval d3 may be variable. That is, the spine immobilization device 1 may be configured such that the interval d3 can be adjusted, by changing the attachment positions of the core parts 22 and 24 to the sheet part 10.

As shown in FIG. 3, the direction in which the core parts 22 and 24 extend is equal to the direction in which a spine 90 extends. That is, the core parts 22 and 24 extend along the spine 90 when the spine immobilization device 1 is worn. In this state, the core parts 22 and 24 laterally support the spine 90. Specifically, the core part 22 supports the spine 90 from one side and the core part 24 supports the spine 90 from the other side. In other words, the core parts 22 and 24 support the spine 90 so as to interpose the spine 90 from both sides, as shown in FIG. 4. Note this diagram shows a similar cross-section to FIG. 2.

The core parts 22 and 24 have greater flexural rigidity in the right-left direction of the body (right-left direction in FIG. 1) than in the front-back direction of the body (direction perpendicular to the page of FIG. 1). Also, the core parts 22 and 24 are deformable in the front-back direction of the body but are not deformable in the right-left direction of the body.

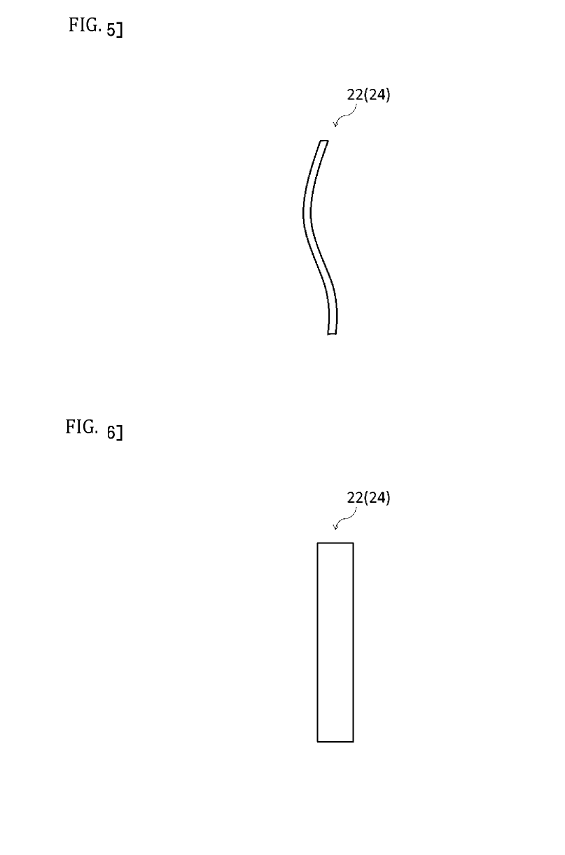

Here, being deformable in the front-back direction of the body means that, when the spine immobilization device 1 is worn, the core parts 22 and 24 freely deform in the front-back direction of the body along the three-dimensional shape of the body surface, as shown in FIG. 5. This diagram shows a state in which the core parts 22 and 24 are seen from the side. Also, not being deformable in the right-left direction of the body means that, when the spine immobilization device 1 is worn, the core parts 22 and 24 maintain their original shape (linear shape in the present embodiment) without deforming in the right-left direction of the body, as shown in FIG. 6. This diagram shows a state in which the core parts 22 and 24 are seen from the front. Plastic, for example, can be used as the material of the core parts 22 and 24.

The effects of the spine immobilization device 1 will be described. The spine immobilization device 1 is able to immobilize the spine, using the core part 20 that laterally supports the spine. That is, the spine is immobilized as a result of movement of the spine in the right-left direction of the body being restricted by the core part 20. Also, the effect of correcting distortion of the spine to the right and left is also obtained.

Moreover, the spine immobilization device 1 can be worn on the body by sticking the sheet part 10 to the body surface. Therefore, the spine immobilization device 1 can be worn, without being wrapped around the body. The area of the body that is covered by the spine immobilization device 1 is thereby narrowly limited, and the sense of discomfort felt when the spine immobilization device 1 is worn can be suppressed to a low level. In actuality in the present embodiment, the spine immobilization device 1 covers only a region of the back near the spine (refer to FIG. 3).

In contrast, in the case of a spine immobilization device that is used by being wrapped around the waist, there are problems such as trousers and skirts being difficult to put on and movement of the entire waist being inhibited, when the spine immobilization device is worn. There is also the problem of stuffiness, particularly heat being exacerbated in summer. The spine immobilization device 1 is able to solve these problems. Also, the spine immobilization device 1 is comparatively compact, and thus is also suitable for carrying. If the user carries the spine immobilization device 1 with them, the spine immobilization device 1 can be put on immediately, in cases such as where lower back pain is experienced while away from home. The spine immobilization device 1 exhibits sufficient effects on mild lower back pain.

The core part 20 consists of the two core parts 22 and 24. Since the spine is thereby supported from both sides, the immobilizing and correcting effects of the core part 20 are enhanced.

The core parts 22 and 24 are fastened to a single one of the sheet part 10. The spine immobilization device 1 can thereby be worn such that the two core parts 22 and 24 are located on both sides of the spine, simply by sticking on a single one of the sheet part 10.

The core part 22 has greater flexural rigidity in the right-left direction of the body than in the front-back direction of the body. That is, the core part 22 flexes readily in the front-back direction of the body but does not flex readily in the right-left direction of the body. Because the core part 22 laterally supports the spine as mentioned above, the core part 22 not readily flexing in the right-left direction of the body is advantageous in firmly immobilizing the spine. The case where the core part 22 is not deformable in the right-left direction of the body is particularly advantageous in firmly immobilizing the spine. On the other hand, the core part 22 readily flexes in the front-back direction of the body, and thus readily deforms along the three-dimensional shape of the body surface. This is advantageous in supporting the curved spine laterally. The case where the core part 22 is deformable in the front-back direction of the body is particularly advantageous in supporting the curved spine laterally. This similarly applies to the core part 24.

In the case where the core parts 22 and 24 are attachable and detachable with respect to the sheet part 10, the spine immobilization device 1 is economical, since the core parts 22 and 24 can be reused, even when replacing the sheet part 10 with a new one, by being reattached to the new sheet part 10. Conversely, the sheet part 10 can also be reused, when replacing the core part 22 and/or the core part 24 with a new one. It is also possible to use the spine immobilization device 1, in a state in which only one of the core part 22 and the core part 24 is removed.

The core parts 22 and 24 laterally support the spine. Therefore, supporting the spine becomes difficult when the surface of the core parts 22 and 24 that opposes the spine is too small. From this point of view, the core parts 22 and 24 preferably have a thickness of 2 mm or more. On the other hand, when the core parts 22 and 24 are too thick, the spine immobilization device 1 tends to become a hindrance when worn. From this point of view, the core parts 22 and 24 preferably have a thickness of 5 mm or less.

In the case where the sheet part 10 has elasticity, the sheet part 10 expands and contracts with movement of the body surface, and thus body movement tends not to be unnecessarily inhibited even when wearing the spine immobilization device 1. Also, in the case where the sheet part 10 has breathability, clamminess tends not to occur in portions covered with the spine immobilization device 1.

In the case where the sheet part 10 can be repeatedly stuck to the body surface, the spine immobilization device 1 is economical, since the spine immobilization device 1 can be used any number of times.

Since the spine immobilization device 1 is for immobilizing the lumbar spine, the core parts 22 and 24 are required to have a certain length. From this point of view, the core parts 22 and 24 preferably have a length of 15 cm or more. Note that the spine immobilization device 1 may be for immobilizing the thoracic spine and/or the sacral spine, in addition to the lumbar spine.

### Second Embodiment

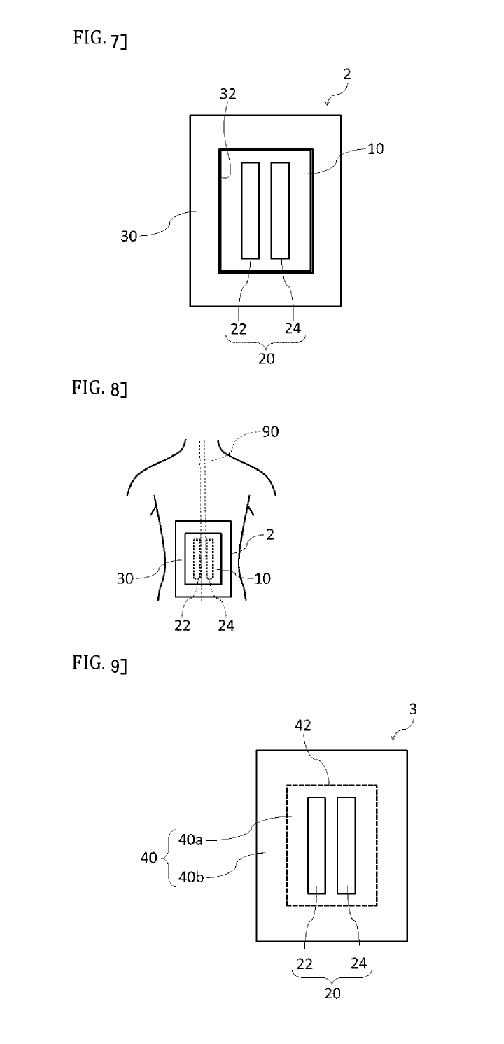

FIG. 7 is a plan view showing a second embodiment of the spine immobilization device according to the present invention. A spine immobilization device 2 is an immobilization device that immobilizes a person's spine, and is provided with a sheet part 30 (large sheet), in addition to the sheet part 10 and the core part 20. The configuration of the sheet part 10 and the core part 20 is as described in the first embodiment.

The sheet part 30 is provided separately from the sheet part 10 and the core part 20. A hole 32 is formed in the sheet part 30. The hole 32 has a size that accommodates the sheet part 10. In the present embodiment, the size and shape of the hole 32 is approximately equal to the size and shape of the sheet part 10. Accordingly, the sheet part 10 can be accommodated within the hole 32 of the sheet part 30 with hardly any gap.

As shown in FIG. 8, the sheet part 30 is stuck to the body surface, such that the sheet part 10 is located within the hole 32. An adhesive having adhesive power on the body surface is applied to the back surface of the sheet part 30. The sheet part 30 preferably can be repeatedly stuck to the body surface. Paper, woven fabric or nonwoven fabric, for example, can be used as the material of the sheet part 30. The material of the sheet part 30 may be the same as the material of the sheet part 10, or may be different. The sheet part 30 preferably has elasticity. Also, the sheet part 30 preferably has breathability.

The effects of the spine immobilization device 2 will be described. The spine immobilization device 2 is able to immobilize the spine, using the core part 20 that laterally supports the spine. Moreover, the spine immobilization device 2 can be worn on the body, by sticking the sheet parts 10 and 30 to the body surface. Therefore, the spine immobilization device 2 can be worn, without being wrapped around the body. The area of the body that is covered by the spine immobilization device 2 is thereby narrowly limited, enabling the sense of discomfort felt when the spine immobilization device 2 is worn to be suppressed to a low level.

The spine immobilization device 2 is provided with the sheet part 30, and the sheet part 30 is stuck to the body surface such that the sheet part 10 is located within the hole 32. Given that the sheet parts 10 and 30 also have a taping function (i.e., function of immobilizing the region to which the sheet parts 10 and 30 are stuck), the area over which this function extends can be expanded by sticking the sheet part 30 to the body surface in addition to the sheet part 10.

The sheet part 30 is provided separately from the sheet part 10. It is thereby possible to leave the sheet part 10 as is and remove only the sheet part 30 from the body surface. Such usage is useful in the case where, for example, lower back pain improves and the area over which the taping function is desired decreases. Also, usages such as leaving the sheet part 10 and replacing the sheet part 30 with a new one or leaving the sheet part 30 and replacing the sheet part 10 with a new one are also possible. Being able to thus replace only one of the sheet parts 10 and 30 is advantageous from an economic perspective and a hygiene perspective.

### Third Embodiment

FIG. 9 is a plan view showing a third embodiment of the spine immobilization device according to the present invention. A spine immobilization device 3 is an immobilization device that immobilizes a person's spine, and is provided with a sheet part 40 and the core part 20. The configuration of the core part 20 is as described in the first embodiment.

The sheet part 40 consists of an inner portion 40a (first portion) and an outer portion 40b (second portion) that is a portion peripheral to the inner portion 40a. The core part 20 is fastened to the inner portion 40a. The inner portion 40a is separable from the outer portion 40b. Specifically, a cutting line 42 is formed around the boundary between the inner portion 40a and the outer portion 40b. The cutting line 42 is a perforation, for example. The inner portion 40a and the outer portion 40b can be separated from each other, by sectioning the sheet part 40 along this cutting line 42. The remaining configuration of the sheet part 40 is similar to the configuration of the sheet part 10 described in the first embodiment.

The effects of the spine immobilization device 3 will be described. The spine immobilization device 3 is able to immobilize the spine, using the core part 20 that laterally supports the spine. Moreover, the spine immobilization device 3 can be worn on the body, by sticking the sheet part 40 to the body surface. Therefore, the spine immobilization device 3 can be worn, without being wrapped around the body. The area of the body that is covered by the spine immobilization device 3 is thereby narrowly limited, enabling the sense of discomfort felt when the spine, immobilization device 3 is worn to be suppressed to a low level.

The sheet part 40 consists of the inner portion 40a and the outer portion 40b that are separable from each other, it is thereby possible to leave the inner portion 40a as is, and remove only the outer portion 40b from the body surface. Also, usages such as leaving the inner portion 40a and replacing the outer portion 40b with a new one or leaving the outer portion 40b and replacing the inner portion 40a with a new one are also possible. Being able to thus replace only one of the inner portion 40a and the outer portion 40b is advantageous from an economic perspective and a hygiene perspective.

### Fourth Embodiment

FIG. 10 is a plan view showing a fourth embodiment of the spine immobilization device according to the present invention. A spine immobilization device 4 is an immobilization device that immobilizes a person's spine, and is provided with a sheet part 50 and a core part 60. In the present embodiment, the spine immobilization device 4 is for immobilizing the cervical spine.

The sheet part 50 has a sheet-like shape and is configured to be stuck to the body surface. Specifically, the sheet part 50 is configured to be stuck to a region of the body surface near the spine (cervical spine). The sheet part 50 is configured to be stuck to the body surface so as to cover the spine. An opening 52 is formed in a portion of the region of the sheet part 50 that covers the spine. The sheet part 50 has, for example, a width (length of the right-left direction in FIG. 10) of about 10 cm and a length (length of the up-down direction in FIG. 10) of about 7 cm. The remaining configuration of the sheet part 50 is similar to the configuration of the sheet part 10 described in the first embodiment.

The core part 60 is fastened to the back surface of the sheet part 50. In the present embodiment, the core part 60 consists of a core part 62 (first core part) and a core part 64 (second core part). The core parts 62 and 64 each have a rectangular columnar shape. The core parts 62 and 64 each have a width d4 from 1 cm to 3 cm inclusive, for example. Also, the core parts 62 and 64 each preferably have a length of 5 cm or more. These core parts 62 and 64 extend parallel at a fixed interval d5. The interval d5 is from 1 cm to 3 cm inclusive, for example. The remaining configuration of the core parts 62 and 64 is similar to the configuration of the core parts 22 and 24 described in the first embodiment.

The effects of the spine in mobilization device 4 will be described. The spine immobilization device 4 is able to immobilize the spine, using the core part 60 that laterally supports the spine. Moreover, the spine immobilization device 4 can be worn on the body, by sticking the sheet part 50 to the body surface. Therefore, the spine immobilization device 4 can be worn, without being wrapped around the body. The area of the body that is covered by the spine immobilization device 4 is thereby narrowly limited, enabling the sense of discomfort felt when the spine immobilization device 4 is worn to be suppressed to a low level.

The opening 52 is formed in the sheet part 50. Given that the spine immobilization device 4 is for immobilizing the cervical spine, a comparatively large spinous process (spinous process of the seventh cervical vertebrae) exists in the cervical spine. When a portion of this spinous process is covered by the sheet part 50, movement of the neck will be inhibited to a large degree. In this respect, it is possible to stick the sheet part 50 to the body surface while avoiding this portion, as a result of the opening 52 being provided. That is, the sheet part 50 need only be stuck to the body surface such that this portion is located within the opening 52.

The spine immobilization device according to the present invention is not limited to the foregoing embodiments, and various variations can be made. In the foregoing embodiments, examples were shown in which two core parts are provided on one sheet part. However, as shown in FIG. 11, the two core parts may be provided on separate sheet pieces. In this diagram, the sheet part consists of two sheet pieces 10a and 10b provided separately. The core part 22 is fastened to one sheet piece 10a and the core part 24 is fastened to the other sheet piece 10b. The configuration of each of the sheet pieces 10a and 10b is similar to the configuration of the abovementioned sheet part 10. The interval between the core parts can thus be freely adjusted, in the case where the two core parts are provided on separate sheet pieces.

In the foregoing embodiments, examples were shown in which two core parts are provided. However, a configuration may be adopted in which only one core part is provided.

In the foregoing embodiments, core parts having a rectangular cross-section (refer to FIG. 2) were illustrated. However, the core part may be recessed in cross-section, as shown in FIG. 12. In this diagram, a groove is formed in the center of a core part 70. The core part 70 supports the spine from both sides by interposing the spine with side surfaces 72 of the groove. This structure enables the spine to be supported from both sides by one core part 70. The spine can also be supported from the back by a bottom surface 74 of the groove, thus enabling the spine to be immobilized more effectively. Note that although an example in which the cross-sectional shape of the groove is a rectangle was shown here, the cross-sectional shape of the groove may be a circular arc.

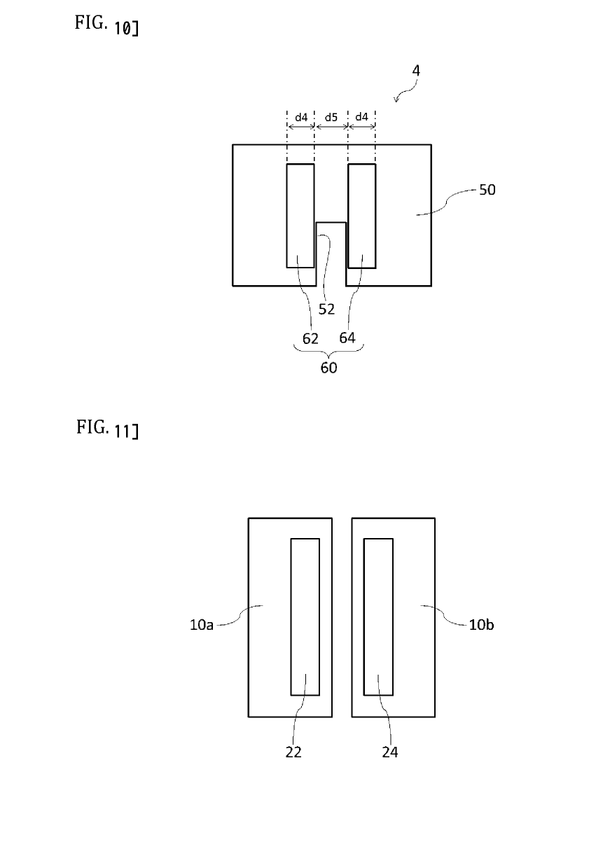

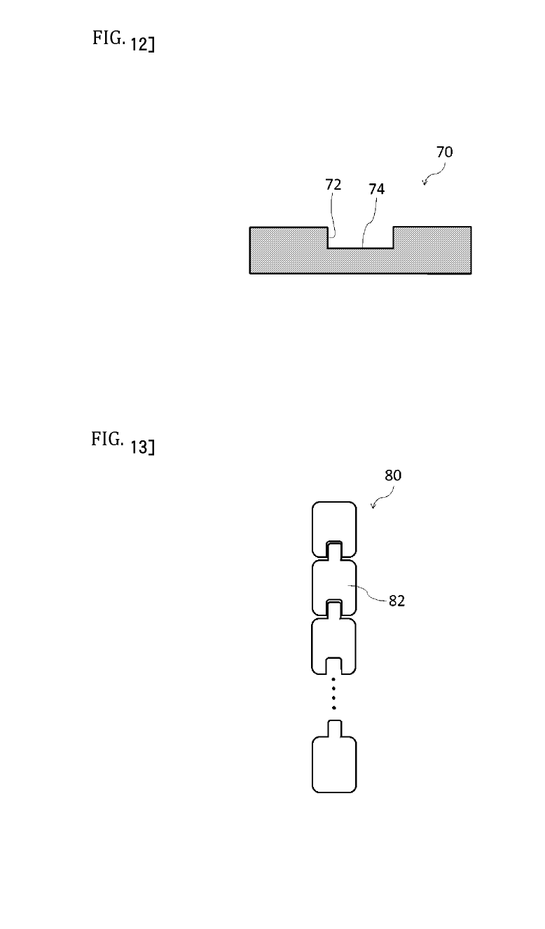

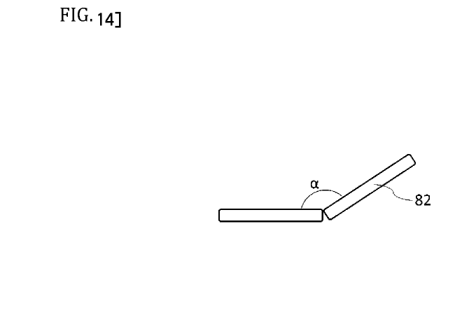

In the foregoing embodiments, core parts that consist of one rod-like member were illustrated. However, a configuration may be adopted in which the core part consists of a plurality of unit parts, as shown in FIG. 13. In this diagram, a core part 80 consists of a plurality of unit parts 82 coupled in the direction in which the core part 80 extends. Adjacent unit parts 82 can turn with respect to each other about an axis parallel to the right-left direction of the body (right-left direction in FIG. 13). Accordingly, this core part 80 is deformable in the front-back direction of the body. Also, the adjacent unit parts 82 are configured to lock when an angle α (0≦α≦180 degrees) formed thereby becomes greater than or equal to a certain value β(e.g., β=165 degrees) as shown in FIG. 14. When locked, the turnable range is restricted to β≦α≦180 degrees.

This structure enables the core part 80 to be folded up, thus improving the convenience with which the spine immobilization device can be stored and carried. Also, since the adjacent unit parts 82 lock when the angle formed thereby becomes greater than or equal to a certain value, the state in which the core part 80 extends along the spine can be maintained when the spine immobilization device is worn.

In the above embodiments, the core part may have shape memory with regard to the front-back direction of the body. In this case, the core part need only be formed using a shape memory material. A shape memory resin, a shape memory alloy, a shape memory nonwoven fabric, or shape memory paper, for example, is given as this material. In the case where the core part thus has shape memory, adhesion of the core part and hence the spine immobilization device to the body surface can be enhanced, by causing the core part to remember the shape along the three-dimensional shape of the body surface.

In the above embodiments, the sheet part may contain a compress solution. In this case, a spine immobilization device that also has a function as a compress is realized. Also, the sheet part may have a function of cooling or warming the body surface. For example, a cooling function can be given to the sheet part, by providing a hydrous gel layer on the back surface of the sheet part. That is, the body surface can be cooled by the evaporation latent heat of the moisture that is contained in the hydrous gel layer.

Alternatively, a warming function can be given to the sheet part, by configuring the sheet part with a dual structure consisting of a front sheet and a back sheet, and providing a heating part between the front sheet and the back sheet. The heating part consists of a heating element containing an oxidizable metal, active carbon and water, for example. The oxidizable metal is a metal that generates oxidation reaction heat. Iron, aluminum, zinc, manganese, magnesium or calcium, for example, can be used as the oxidizable metal.

### LIST OF REFERENCE NUMERALS

1 spine immobilization device

2 spine immobilization device

3 spine immobilization device

4 spine immobilization device

10 sheet part

20 core part

22 core part (first core part)

24 core part (second core part)

30 sheet part (large sheet)

32 hole

40 sheet part

40a inner portion (first portion)

40b outer portion (second portion)

42 cutting line

50 sheet part

52 opening

60 core part

62 core part (first core part)

64 core part (second core part)

70 core part

72 side surface of groove

74 bottom surface of groove

80 core part

82 unit part

90 spine

1. A spine immobilization device for immobilizing a spine of a person, comprising:
a sheet part configured to be stuck to a body surface; and
a core part configured to be fastened to the sheet part and to extend along the spine so as to laterally support the spine.

2. The spine immobilization device according to claim 1,
wherein the core part includes:
a first core part configured to support the spine from one side; and
a second core part configured to support the spine from the other side.

3. The spine immobilization device according to claim 2, wherein the first and second core parts are fastened to a single one of the sheet part.

4. The spine immobilization device according to claim 2,
wherein the sheet part includes first and second sheet pieces provided separately, and
the first and second core parts are respectively fastened to the first and second sheet pieces.

5. The spine immobilization device according to claim 1, wherein the core part has a recessed shape in cross-section, and supports the spine from both sides by interposing the spine.

6. The spine immobilization device according to claim 1, wherein the core part has greater flexural rigidity in a right-left direction of a body than in a front-back direction of the body.

7. The spine immobilization device according to claim 6, wherein the core part is deformable, in the front-back direction of the body.

8. The spine immobilization device according to claim 7,
wherein the core part consists of a plurality of unit parts coupled in a direction in which the core part extends, and
the unit parts that are adjacent are turnable with respect to each other about an axis parallel to the right-left direction of the body.

9. The spine immobilization device according to claim 6, wherein the core part is not deformable in the right-left direction of the body.

10. The spine immobilization device according to claim 1, wherein the core part has shape memory with regard to a front-back direction of a body

11. The spine immobilization device according to claim 1, wherein the core part is attachable and detachable with respect to the sheet part.

12. The spine immobilization device according to claim 1, wherein the sheet part contains a compress solution.

13. The spine immobilization device according to claim 1, wherein the sheet part has a function of cooling the body surface.

14. The spine immobilization device according to claim 1, wherein the sheet part has a function of warming the body surface.

15. The spine immobilization device according to claim 1, comprising a large sheet having a hole of a size that accommodates the sheet part,
wherein the large sheet is configured to be stuck to the body surface, such that the sheet part is located within the hole.

16. The spine immobilization device according to claim 1,
wherein the sheet part consists of a first portion to which the core part is fastened and a second portion that is a portion peripheral to the first portion, and
the first portion is separable from the second portion.

17. The spine immobilization device according to claim 1, wherein the sheet part is repeatedly stickable to the body surface.

18. The spine immobilization device according to claim 1, wherein the spine immobilization device is for immobilizing a lumbar spine.

19. The spine immobilization device according to claim 1, wherein the spine immobilization device is for immobilizing a cervical spine.

20. The spine immobilization device according to claim 19,
wherein the sheet part is configured to be stuck to the body surface so as to cover the spine, and
an opening is formed in a portion of a region of the sheet part that covers the spine.
